(19) United States
(12) Patent Application Publication
(10) Pub. No.: US 2008/0090298 A1
(43) Pub. Date: Apr. 17, 2008

(54) Method and system for identification of protein-protein interaction

(76) Inventor: James Alexander Apffel, Mountain View, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/580,840

(22) Filed: Oct. 16, 2006

(51) Int. Cl. G01N 33/00

(52) U.S. Cl. 436/86; 436/173

(57) ABSTRACT

A method for the characterization of protein-protein interactions based on diagonal mass spectrometry is provided. Proteomic samples containing interacting proteins are chemically crosslinked either in vivo or in vitro. After a high resolution chromatographic separation, crosslinked interacting proteins are introduced directly into a mass spectrometer. During the data acquisition, the mass spectrometer alternates between two discrete acquisition states. In the first acquisition state, the crosslinked complexes are analyzed. In the second acquisition state, the crosslinking is cleaved and the mass spectra of the dissociated proteins are collected. Following the data acquisition, the raw mass spectral data is deconvoluted and reconstructed into a diagonal MS plot of crosslinked proteins vs. component proteins to explore protein-protein interactions.

### TECHNICAL FIELD

The invention relates generally to protein analysis methods and more particularly to rapid and high resolution detection and identification of protein-protein interaction using diagonal mass spectrometry (MS) analysis.

### BACKGROUND OF THE INVENTION

Protein-protein interactions constitute an important part of the molecular mechanism of biological processes. One method for detecting protein-protein interactions is diagonal gel electrophoresis (see e.g., Brennan et al., J Biol Chem 2004, 279:41352-41360). In this technique, interacting proteins are cross-linked in vivo or in vitro, usually using disulfide formation between cysteines. The mixture, containing crosslinked complexes is then separated by size with a first dimension sodium dodecyl sulfate polyacrylamide gel electrophoresis (SDS-PAGE). The disulfide bonds are then reduced and the mixture is re-separated by size with SDS-PAGE. In the second dimension of separation, all components that were originally single proteins, unassociated with any complex, migrate the same as in the first dimension, forming a diagonal pattern in the two-dimension (2D) separation. The components of the complexes that were originally bound together are now unbound and will migrate independently, off the diagonal. Conceptually, this approach sounds relatively simple and elegant. However, it suffers from a number of specific drawbacks that have resulted in low adoption rate. In practice, the use of gel electrophoresis has been limited in terms of resolution and the information produced directly from the electrophoresis experiment has been insufficient to identify the interacting proteins and requires additional analytical steps for identification. Furthermore, limitations inherent to gel electrophoresis such as sample solubility, speed and automation issues still hamper the usefulness of this approach.

Mass spectrometry has been applied to the characterization of protein-protein interactions. However, the characterization has typically been carried out under extremely well controlled and constrained systems in which a single protein complex was highly purified or expressed in a purified form and isolated (see e.g., Videler et al., FEBS Lett 2005, 579:943-947; Stenberg et al., J Biol Chem 2005, 280:34409-34419; Sobott et al., Philos Transact A Math Phys Eng Sci 2005, 363:379-389; discussion 389-391; and Benesch et al., Anal Chem 2003, 75:2208-2214).

The combination of mass spectrometry and in vitro chemical crosslinking has also been used for characterization of protein-protein interactions at the peptide level (see e.g., Rappsilber et al., Anal Chem 2000, 72:267-275; Back et al., Anal Chem 2002, 74:4417-4422; and Trester-Zedlitz et al., J Am Chem Soc 2003, 125:2416-2425). More frequently, this approach has been applied to structural characterization of proteins by analysis of intra-molecular crosslinking (see e.g., Young et al., Proc Natl Acad Sci USA 2000, 97:5802-5806; Back et al., J Mol Biol 2003, 331:303-313; Collins et al., Bioorg Med Chem Lett 2003, 13:4023-4026; Dihazi et al., 2003, 17:2005-2014; Schulz et al., Biochemistry 2004, 43:4703-4715; Sinz et al., Anal Bioanal Chem 2005, 381:44-47). Typically, following crosslinking and isolation, the proteins and complexes are proteolytically digested and the fragments are analyzed by mass spectrometry. The data obtained can be used to infer the identity of the proteins involved in the interaction and the sites of interaction. However, detailed information about the proteins character such as sequence modifications or presence of post translational modifications (PTMs) is lost in this approach.

Another approach to protein-protein interaction characterization by mass spectrometry is tandem affinity probes mass spectrometry (TAP-MS) (see e.g., Gavin et al. Nature 2002, 415:141-147). In this approach, a “bait” protein is expressed with two affinity probes expressed as part of its sequence, in vivo. Following its interactions in normal biological milieu, the bait protein forms complexes with other proteins. The complexes are purified through two successive orthogonal stages of affinity purification and the purified protein complexes are characterized by digestion and peptide level analysis by mass spectrometry. Although this approach has the potential to be competitive with the more standard approach of the yeast two hybrid (Y2H) system, similar to Y2H, it requires costly or time consuming experimental preparations, such as the preparation of specific antibodies, genetic constructs or protein translation systems to characterize interactions of specific target-bait interactions.

Therefore, the need remains for a cost effective assay method that can quickly detect and identify multiple protein complexes with high resolution.

### SUMMARY OF THE INVENTION

One aspect of the present invention relates to a method for identifying protein-protein interactions. The method comprises: crosslinking interacting proteins; subjecting crosslinked proteins to a liquid chromatographic separation; alternatively subjecting an effluent of the liquid chromatographic separation to mass spectrometry analysis for molecular weight determination of intact proteins and protein complexes in a first state and a second state, wherein in the first state, the effluent is analyzed under conditions that preserve crosslinks and, wherein in the second state, the effluent is analyzed under conditions that disrupt crosslinks; and identifying components of a protein complex by plotting molecular weight data of the first state versus molecular weight data of the second state.

In an embodiment, the method further comprises collecting fractions from the liquid chromatographic separation; subjecting the fractions of interest to a peptide level mass spectrometry analysis; and identifying components of the protein complex by integrating data from the mass spectrometry analysis for molecular weight determination of intact proteins and protein complexes, and data from the peptide level mass spectrometry analysis.

In another embodiment, the method further comprises the step of: prior to peptide level mass spectrometry analysis, selecting fractions of interest based on results obtained by plotting molecular weight data of the first state versus molecular weight data of the second state.

In another embodiment, the peptide level mass spectrometry analysis is a bottom-up LC-MS/MS analysis or an matrix assisted laser desorption ionization mass spectrometry (MALDI MS) analysis.

In another embodiment, the method further comprises isolating and concentrating a sub-proteomic fraction of crosslinked proteins prior to the liquid chromatographic separation.

In another embodiment, the liquid chromatographic separation is performed with a macroporous reverse phase material.

In another embodiment, the mass spectrometry analysis for molecular weight determination is performed with electrospray ionization time-of-flight mass spectrometry (ESI-TOF MS) or MALDI-TOF MS.

In yet another embodiment, crosslinks of the crosslinked protein are disrupted by a gas phase fragmentation method selected from the group consisting of collisionally induced dissociation (CID), IR Multiphoton Dissociation (IRMPD), Electron Transfer Dissociation (ETD), Electron Capture Dissociation (ECD), Metastable Ion Dissociation (MAID) and Surface Induced Dissociation (SID).

Another aspect of the present invention relates to a system for identifying protein-protein interactions. The system comprises a liquid chromatographic unit capable of high resolution separation of protein molecules; a mass spectrometry (MS) unit coupled to the chromatographic unit for alternatively determining molecular weights of intact proteins and protein complexes in an effluent of the liquid chromatographic unit under a first state and a second state, wherein in the first state, the effluent is analyzed under conditions that preserve crosslinks and, wherein in the second state, the effluent is analyzed under conditions that disrupt crosslinks; and a data acquisition system capable of collecting a first state MS data and a second state MS data, plotting the first state MS data versus the second state MS data to detect components of a protein complex.

In an embodiment, the system further comprises a second MS unit for peptide based-identification of proteins in chromatographic fractions.

In another embodiment, the data acquisition system is capable of collecting protein ID data from the second MS unit and integrating the first MS state data, the second MS state data, and the protein ID data to identify components of the protein complex.

### DETAILED DESCRIPTION OF DRAWINGS

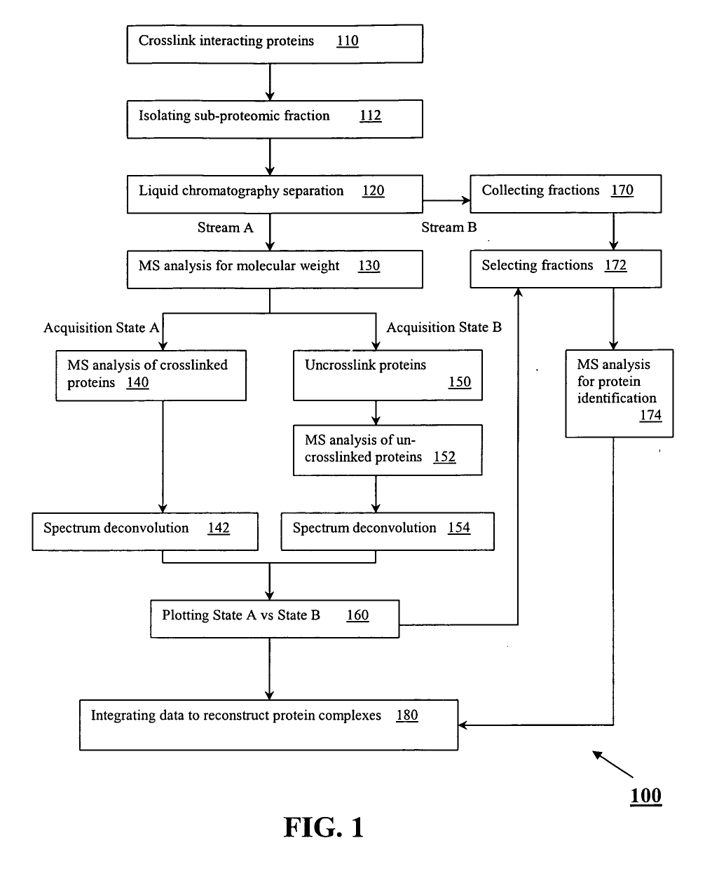

FIG. 1 is a block diagram showing an embodiment of the diagonal MS method for identification of protein-protein interactions.

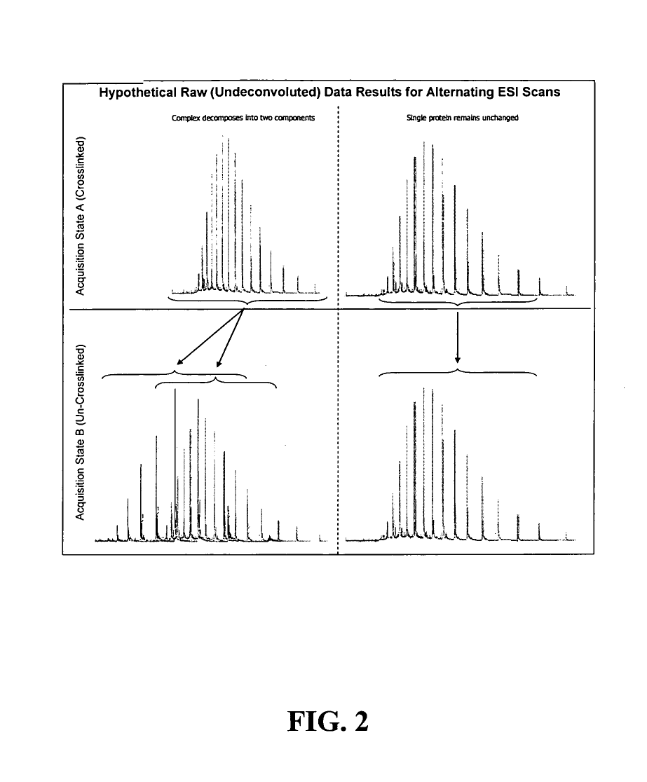

FIG. 2 shows hypothetical raw (undeconvoluted) data from alternating ESI scans.

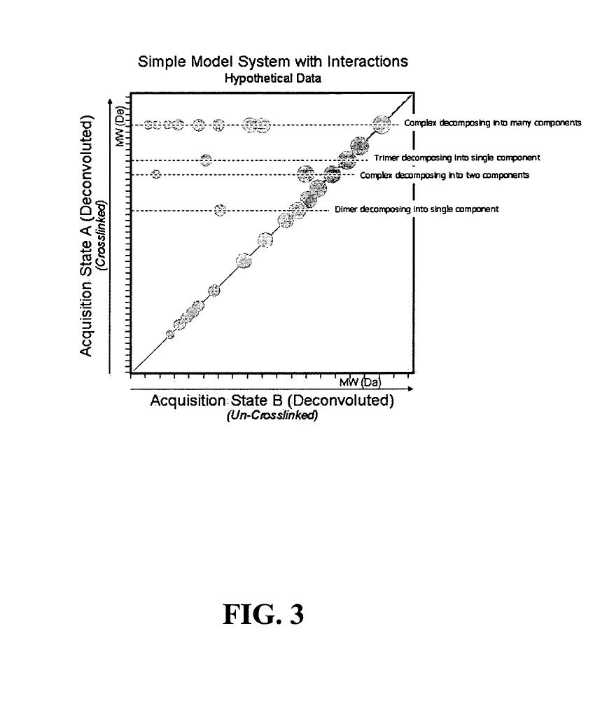

FIG. 3 is a schematic showing the hypothetical result of diagonal MS of proteins with interactions.

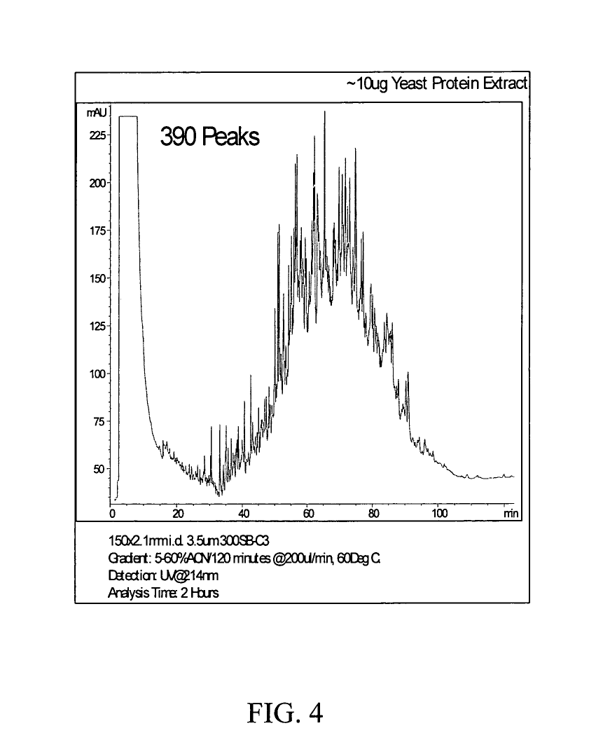

FIG. 4 is a representative chromatogram showing the resolution of reversed phase chromatography.

### DETAILED DESCRIPTION OF THE INVENTION

A method for the characterization of protein-protein interactions based on diagonal mass spectrometry analysis is provided. Initially proteomic samples containing interacting proteins are crosslinked either in vivo or in vitro. After a high resolution chromatographic separation, separated proteins and protein complexes are introduced directly into a mass spectrometer for determination of their molecular weights. During the data acquisition, the mass spectrometer alternates between two discrete acquisition states. In the first acquisition state, the crosslinked complexes are analyzed. In the second acquisition state, the crosslinking is cleaved and the mass spectra of the dissociated proteins are collected. Following the data acquisition, the raw mass spectral data is deconvoluted and reconstructed into a diagonal MS plot of crosslinked proteins vs. component proteins which can be interpreted to explore protein-protein interactions.

FIG. 1 shows an embodiment of the diagonal MS method 100 of the present invention. In the method 100, interacting proteins are crosslinked with a crosslinking reagent (step 110). A crosslinked protein sample is then subjected to a high resolution liquid chromatographic analysis and the effluent flow from the chromatographic column is split into two streams (Stream A and Stream B, step 120). Effluent flow from Stream A is introduced into a MS unit with alternating acquisition states (step 130). In acquisition state A, effluent flow from the chromatographic column is directly introduced into an ion source for MS analysis of crosslinked proteins (step 140). In acquisition state B, the effluent flow is first treated to un-crosslink proteins in the effluent flow (step 150) and then subjected to MS analysis for un-crosslinked proteins (step 152). Data from alternating acquisitions are collected into separate file channels, deconvoluted (steps 142 and 154), and plotted to generate protein complex component data (step 160). Effluent from Stream B is collected in fractions (step 170). Based on the outcome of step 160, key fractions are selected (step 172) and subjected to further MS analysis to produce identification data for proteins in these fractions (174). Finally, the protein complex component data (from step 160) and the identification data (from step 174) are integrated to reconstruct protein complexes (step 180).

### Crosslinking

The crosslinking step 110 may be performed in vitro or in vivo. In one embodiment, the crosslinking is performed in vitro. This procedure involves the formation of covalent bonds between two proteins by using bifunctional reagents containing reactive end groups that react with functional groups, such as primary amines and sulfhydryls, of amino acid residues. If two proteins interact with each other, they can be covalently crosslinked. The formation of crosslinks between two distinct proteins is a direct evidence of their close proximity.

A wide range of crosslinking reagents are commercially available from major suppliers such as Pierce (Rockford, Ill.), Molecular Probes (Eugene, Oreg.), and Sigma (St. Louis, Mo.). The crosslinking reagents can be either homo- or hetero-bifunctional reagent with identical or non-identical reactive groups, respectively. The homo-bifunctional reagents have the advantage of speed and simplicity since a single step reaction is required. However, at high protein concentrations, homo-bifunctional reagents may result in intramolecular crosslinking and the formation of multimers. The hetero-bifunctional reagents have the advantage of being more selective towards directly interacting proteins. However, the use of hetero-bifunctional reagents requires multi-step reactions and the second step is often photo-initiated, adding to the complexity of the sample preparation.

The reactivity of the crosslinking reagent should be general enough to crosslink all reacting proteins but not too general (e.g., a homo-bifunctional reagent directed towards amines) so as to increase the possibility of intramolecular crosslinking. The reagent should not overly perturb the mass spectral behavior of the proteins. For example, an amine reactive reagent that capped all amino groups on a protein without replacing the charge would drastically alter the electrospray ionizability of the protein, and is hence undesirable. In one embodiment, the crosslinking reagent is a hetero-bifunctional reagent with reactive groups directed towards functional moieties of intermediate availability.

The length of the bridge between the interacting proteins will play an implicit role in selectivity towards what interactions are detected. Therefore, crosslinking reagents may use spacer arms of various length, typically between 5 and 20 Å. Optimal arm length can be determined experimentally.

Examples of homo-bifunctional crosslinking reagents include, but are not limited to, glutaraldehyde, imidoesters such as dimethyl adipimidate (DMA), dimethyl suberimidate (DMS), and dimethyl pimelimidate (DMP) with spacer arms of various lengths between the reactive end groups. In one embodiment, the crosslinking reagent is a reversible homo-bifunctional crosslinkers. Examples of reversible home-bifunctional crosslinkers include, but are not limited to, N-hydroxysuccinimide (NHS) esters such as dithiobis(succinimidylpropionate) (DSP), and dithiobis(sulfosuccinimidylpropionate) (DTSSP), and Bis[2-(Succinimidooxycarbonyloxy)ethyl]sulphone (BSOCOES). These crosslinkers can be cleaved by treatment with thiols, such as β-mercaptoethanol or dithiothreitol.

Examples of hetero-bifunctional crosslinkers include, but are not limited to, hetero-bifunctional crosslinkers having one amine-reactive end and a sulfhyfryl-reactive moiety, hetero-bifunctional crosslinkers having a NHS ester at one end and an SH-reactive group, such as maleimide or pyridyl disculfide, at the other end; and hetero-bifunctional crosslinkers having a photoreactive group, such as Bis[2-(4-azidosalicylamido)ethyl]disulfide (BASED).

In one embodiment, the crosslinking reagent is sulfo-SFAD (Sulfosuccinimidyl-[perfluoroazidobenzamido]ethyl-1,3′-dithiopropionate) (Pierce Chemical, Rockford, Ill.). Sulfo-SFAD is a hetero-bifunctional crosslinking reagent. Exposed amine groups in proteins can be reacted with the NHS-Ester moiety of the reagent. The crosslinking can also be initiated through photoconjugation by radiation at 320 nm for reaction with a halogen substituted phenylazide group at the other end. The two reactive groups are joined by a cleavable disulfide linkage, so the crosslinking can be reversed by reduction. The reagent is water soluble, couples with high efficiency and has a spacer arm of approximately 15 Å in length.

In another embodiment, the crosslinking reagent is a hetero-trifunctional crosslinking reagent having two reactive groups that can be used to crosslink interacting proteins and a third reactive group (e.g., biotin) that can be used as a selective isolation group (e.g., for streptavidin pull-down). In this embodiment, the affinity portion of the crosslinking reagent is used to selectively isolate only those proteins that were involved in chemical crosslinking reactions. Non-interacting proteins would be washed away and would not be subjected to the first dimension separation.

The crosslinking reagent can be hydrophobic or hydrophilic. If the proteins of interest are cytosolic proteins, a hydrophilic crosslinking reagent may be used so that the crosslinking reagent can be introduced into cellular milieu without perturbing existing interactions. If the proteins of interest are membrane proteins, hydrophobic crosslinking reagents may be used.

In another embodiment, the crosslinking step 110 is performed in vivo. In vivo crosslinking offers the advantage of capturing both stable and transient interactions in a biologically relevant context with a minimal perturbation to the system under study. In vivo crosslinking would effectively take a snapshot of the system at a given point in time. However, in vivo crosslinking requires that the crosslinking reagent be cell permeable, the crosslinking can be initiated, and the crosslinking reaction be reversible. Examples of in vivo crosslinking reagents include, but are not limited to, formaldehyde and BSOCOES.

### Liquid Chromatography (LC)

A sample of crosslinked proteins is prepared for high resolution LC separation. The sample typically contains a mixture of individual proteins (which are not crosslinked to each other) and protein complexes with individual components crosslinked to each other. As shown in FIG. 1, an optional isolation step 112 may be added at this stage to isolate and concentrate the sub-proteomic fraction of interest. For example, if the protein complexes of interest are known to be located in the endoplasmic reticulum (ER), the sample can be enriched for the ER fraction by density gradient separation. Alternatively, if a hetero-trifunctional crosslinking reagent with biotin as a selective isolation group is used, the crosslinked proteins can be isolated by a streptavidin pull-down.

The high resolution liquid chromatographic separation (step 120) can be carried out using high performance liquid chromatography (HPLC), fast protein liquid chromatography (FPLC) or other comparable high resolution liquid chromatographic techniques. In one embodiment, the first dimension chromatography is performed using macroporous reversed phase (mRP) HPLC columns because of their high resolution, high recovery and potentially high speed. Chromatographic conditions, such as stationary phase, mobile phases, elution gradient, temperature, flow rate, etc. are determined based on the sample content and the characteristics of the proteins of interest. One skilled in the art would recognize that a range of chromatographic modes can be used in the method 100.

The chromatographic conditions should be selected in favor of high resolution. For a given sample complexity, the resolution is directly related to the speed of the separation. Since the entire analysis is completed in the time scale of a chromatographic separation, relatively long separations with long chromatographic gradients can be used.

The dimensions of the chromatographic column are selected based on the sensitivity of the system and the amount of sample available. Since subsequent peptide level MS analysis of collected fractions may be required for positive protein identification, the chromatographic scale needs to be large enough to support a split flow. On the other hand, the ionization process of the subsequent MS analysis is a concentration sensitive phenomenon. For a fixed sample amount, a small column will result in increased peak concentration and, consequently, increased sensitivity of the MS analysis. In one embodiment, capillary scale columns (300-500 μm i.d.) are used. These columns can be operated at 4-10 μL/min flow rate with on-line UV/VIS detection, microfraction collection and nanospray or Chip HPLC ESI-MS flowing at 200 nL/min. In another embodiment, the liquid chromatographic analysis is performed with a column having a retentive stationary phase, such as a macroporous reverse phase column. Columns with retentive stationary phase allow large volumes of sample to be injected without band broadening.

The complexity of mixtures that can be dealt with by the present invention will largely depend on the resolution of the chromatographic separation in step 120. This, in turn, has ramifications in terms of separation speed and total analysis time. The limit of the maximum number of protein complexes that might be separated by the chromatographic system depends on the chromatographic modes and conditions utilized. In one embodiment, In one embodiment, the chromatographic analysis of the present invention resolves 100-150 proteins in 30-60 minutes using a reversed phase chromatographic material. As shown in FIG. 4, reversed phase chromatography is capable of resolving nearly 400 peaks in 90 minutes from a complex proteomic sample of intact proteins.

### LC/MS Interface

The effluent of the LC is split into two streams for the MS analysis of intact proteins (Stream A) and peptide identification (Stream B). A post-column, post-UV detector, pre-fraction collector split can be easily achieved with low dead volume splitters that are commercially available. In one embodiment, the LC effluent in stream A is directly coupled to the ion source of the mass spectrometer. In this case, the chromatographic conditions may be adjusted to be compatible with the subsequent MS analysis. For example, best chromatographic resolution for proteins is typically obtained with a mobile phase containing approximately 0.1% trifluoroacetic acid (TFA), which is known to suppress electrospray ionization efficiency. Formic acid may be used to substitute TFA, but it may result in reduced chromatographic resolution and performance. In one embodiment, the mobile phase is composed of 0.1% formic acid and 0.01% TFA.

As previously mentioned, the MS analysis in Stream A is performed in two alternating acquisition states. In acquisition state A, effluent flow from the chromatographic column is directly introduced into an ion source for MS analysis of crosslinked proteins (step 140). In acquisition state B, the effluent flow is first subjected to a reaction to cleave the crosslink (step 150) and is then analyzed by the MS for un-crosslinked proteins (step 152).

In one embodiment, the alternating scan functionality of the MS is synchronized with the reaction chemistry through split flow reactors or segmented flow reactors. For a split flow reactor, the LC flow is split 50:50. Half of the flow is introduced into the ionization source without modification, while the other half is subjected to a reaction to cleave the crosslink. The two flows are selectively introduced into the mass spectrometer by an alternating selection valve or through a spray multiplexer. For a segmented flow reactor, the LC effluent is introduced into a reaction capillary with an immiscible separating liquid to generate discrete volume segments that are physically separated from each other. The crosslink cleavage reaction is generated in alternate segments while intact complexes are maintained in the rest. Thus, a train of alternating segments containing complexes and dissociated components is generated. The entire flow is introduced into the MS and the acquisition states synchronized with the flow segmentation.

In another embodiment, a post-column reaction system is employed to perform the un-crosslinking step 150. Depending on the crosslinking reagent used in step 110, the post-column reaction system may use a number of chemical or physical methods to induce crosslink cleavage. For example, if a crosslinking reagent utilizing a disulfide linkage is employed in step 110, the disulfide linkage can be cleaved by reaction with a reducing agent such as dithiothrietol (DTT). If formaldehyde is used as the crosslinking reagent, the crosslinking can be reversed thermally by introducing a thermal reactor into a split flow reaction scheme. If a photosensitive crosslinking reagent is used in step 110, the crosslinking can be cleaved with a pulsed light source.

The un-crosslinking may also be performed using any of the fragmentation methodologies that are used in an MS/MS type instrument. These could include a wide range of complimentary techniques, such as collisionally induced dissociation (CID), infrared multiphoton dissociation (IRMPD), electron transfer dissociation (ETD), electron capture dissociation (ECD), metastable ion dissociation (MAID) or surface induced dissociation (SID) (See e.g., Nielsen et al., Mol Cell Proteomics 2005, 4:835-845). The un-crosslinking may be performed in the ionization source or in a separate chamber outside the ionization source. If a fragmentation method is used for the un-crosslinking step, the crosslinking reagent should be sufficiently stable to withstand the ionization process, but more labile than any of the protein bonds themselves, such that the crosslinks are the first bonds to be broken in the fragmentation process.

In one embodiment, CID is used to disrupt protein-protein crosslinking in an electrospray ion source using a technique called In-Source CID (Bristow et al., Rapid Communications in Mass Spectrometry 2002, 16:2374-2386; and Bure et al., Current Organic Chemistry 2003, 7:1613-1624). In CID, labile molecules or complexes are electrostatically accelerated in a relatively high pressure region of a mass spectrometer. The ions undergo collisions with the surround gas (usually Nitrogen, Helium or Argon) and the energy imparted to the target molecule due to collision results in fragmentation of the molecule or complex. These collisions are ergodic, which is to say the energy is uniformly distributed through the molecular structures and the fragmentation patterns depend on the molecular stability.

### Ionization

Any ionization technique capable of generating useful and interpretable spectra for high molecular weight complexes and components can be used in the present invention. The ionization technique should be a gentle ionization method which will not cause degradation to the proteins being analyzed. Since many of the protein complexes may be present at low levels, the ionization technique needs to be optimized for sensitivity. If the LC is directly coupled to the MS, the ionization technique also needs to be able to handle direct and continuous introduction of effluent from liquid phase separation.

Among the ionization techniques currently available, electrospray ionization satisfies all the above-described requirements. Other ionization techniques, such as Atmospheric Pressure Chemical Ionization (APCI), Fast Atom Bombardment, Direct Liquid Introduction or Thermospray may also be used in the present invention. In one embodiment, a high resolution, macroporous reversed phase (mRP) column is directly coupled with electrospray ionization. In another embodiment, an LC column with nanoscale flows is coupled with electrospray ionization. Given limited sample quantities, nanoscale separations are more sensitive then the use of conventional diameter columns.

As discussed above, gas phase fragmentation may be employed to un-crosslink proteins in the ionization source. In one embodiment, the crosslinking reagent is designed such that the crosslink can be disrupted by gas phase dissociation. The un-crosslinking efficiency is controlled through manipulation of collision gas pressures and excitation energy. Since the present invention does not require any type of parent selection, MS/MS capability is not required. However, in one embodiment, the collision cell of a QTOF is used to conduct CID on alternate scan acquisitions. The MS/MS capability is used to reject specific mass ranges as “noise”. In another embodiment, a linear ion trap (LIT)-TOF instrument is used for gas phase fragmentation and the fragmentation process is synchronized with a chromatographic time scale. In a linear ion trap, analyte molecules can be stored in a gas phase trap and manipulated with gas-phase reaction chemistry to induce specific fragmentation and charge state manipulation. Following these manipulations, the resulting ions can be analyzed by TOF-MS with high mass accuracy and resolution. The use of LIT-TOF allows more complete control and greater options for gas phase ion-ion chemistry, and hence provides greater flexibility in design and choice of a crosslinking reagent.

### Mass Analyzer

The mass analyzer of the present invention can be any mass analyzer with a wide mass range capability for capturing the full possibilities of multiple charged ion distributions for large molecular weight complexes. The mass analyzer should have a high mass accuracy for calculating the deconvoluted molecular weight of intact proteins and complexes, and a high resolution to capture as much detail in isotopic distributions as possible for the individual charge states. The mass analyzer also need a high transmission efficiency, a wide detection dynamic range for detecting low abundance protein complexes in the presence of high abundance background proteins, and fast acquisition times to allow cycling between acquisition state A and acquisition state B on a chromatographic time scale while collecting sufficiently large numbers of transients to maintain high sensitivity and spectral fidelity. In one embodiment, the mass analyzer is a time-of-flight mass spectrometer (TOF-MS). In another embodiment, the mass analyzer is a 3D Ion Trap, a Fourier Transform Mass Spectrometer (FTMS), a Linear Ion Trap (LIT), an Orbitrap or an Ion Cyclotron Resonance Mass Spectrometer (ICR-MS).

As shown in FIG. 1, following the LC separation, the LC effluent is split and effluent in Stream B is collected in fractions for subsequent peptide analysis (step 170). In one embodiment, a conventional (4.6 mm i.d.) or narrow (2.1 mm i.d.) bore mRP column is used in the method 100. A vast majority (99%+) of the effluent is collected in Stream B with a fraction collector while a very small proportion at 1-5 μL/min is funneled into Stream A and is introduced via nanospray or a ChipMS infusion chip directly into a ESI-TOF MS. Depending on the application and sample load, it may be necessary to use smaller bore columns in other embodiments to maximize peak concentration and sensitivity.

In another embodiment, off-line matrix assisted laser desorption ionization-MS (MALDI-MS) is used as the mass analyzer. Fractions are collected off-line after the LC separation or spotted directly onto MALDI plates for subsequent analysis. Depending on the number of fractions and/or spots, the resolution of the chromatographic separation could be maintained to a greater or lesser degree. MALDI generally generates singly charged ions rather than the multiply charged ion distributions found in electrospray. For this reason, implementation of this approach would require use of a high mass capable, TOF mass analyzer.

### Data Analysis

The initial raw data consist of a set of chromatographic signals from the detector that monitors the separation and two synchronized but separable file channels of mass spectral data for each of the MS acquisition states (i.e., acquisition of data from crosslinked samples and acquisition of data from un-crosslinked samples). In one embodiment, the initial mass spectral data consists of multiply charged ion distributions typical of electrospray ionization of intact proteins. A hypothetical example of what this data might look like is shown in FIG. 2. On the right is the example of an intact protein that is not a member of a complex. Thus its spectrum is identical under the two acquisition states (crosslinked vs un-crosslinked). After deconvolution of this spectrum to yield an intact molecular ion, the data would fall on the diagonal of a plot of State A vs State B as shown in FIG. 3. As a second example, the spectra on the left of FIG. 2 represent those of a two component protein interaction. The spectrum on the top of the intact complex would deconvolute to a high molecular weight component while after decomposition of the crosslinking, two separate ion distributions would be deconvoluted into two smaller protein components. These would be represented in FIG. 3 by the spots annotated “Complex decomposing into two components”. Assuming ideal performance, the masses would be additive and the stoichiometry of the interaction could be determined from the data.

The MS molecular weight data may not be specific enough to generate a definitive protein identification for the individual components. For this reason, the chromatographic flow is split into Stream A and Stream B. Following the data analysis of the intact proteins in Stream A, fractions in Stream B can be identified for subsequent peptide level MS/MS analysis. In one embodiment, the peptide analysis is performed with a nano LC-MS/MS system. The protein identification may be facilitated by database searching. In another embodiment, the database search is performed using a SpectrumMill® software (Millennium Pharmaceuticals, Cambridge, Mass.).

The sequencing data obtained from the peptide MS analysis adds confidence to protein identification. For example, the molecular weight data from the whole molecule MS analysis (Stream A) provides information on the intact protein (including post-translation modifications (PTM's)), whereas the peptide based MS analysis (Stream B) shows molecular weights based on amino acid sequences alone. Thus inferences can then be made about the character and nature of the PTMs based on the difference between the whole molecule MS analysis and peptide MS analysis. These inferences can be further investigated from the raw peptide MS/MS data directly.

The ability to associate interacting components to the complex with which they are associated will be limited by the resolution of the system. For example, if two complexes co-elute in LC, then upon cleavage of the crosslinks, the individual components will have to be assigned to the appropriate complex. If the molecular weights of the complex and each individual component can be determined with high precision, reconstitution should not be a problem. For example, if a 60 kD complex is associated with four components of 50 kD, 35 kD, 25 kD, and 10 kD, it would be clear that the original complex is a mixture a of two different 60 kD complexes: one consists of the 50 kD and 10 kD components, while the other one consists of the 35 kD and 25 kD components.

This challenge can be further simplified by initial sample preparation to selectively isolate protein complexes from irrelevant matrix components. In one embodiment, the crosslinking reagent includes an affinity tag and the crosslinked proteins are selectively isolated from a mixture. In another embodiment, the proteins of interest are isolated by affinity methods following crosslinking. In yet another embodiment, sub-cellular fractions containing the proteins of interest are isolated prior to liquid chromatography.

The method of the present invention may be implemented in a miniaturized or microfluidic format in order to minimize the quantity of samples required for the protein complex analysis. In one embodiment, the detection system uses an UV/VIS detector and is capable of performing an analysis with 1-10 ng of protein. In another embodiment, the detection system uses mass spectrometry as an on-line detector and is capable of performing an analysis with proteins in the range of sub-femto moles. The detection scale and capacity can be adjusted for each application such that enough original material can be introduced and separated by the system to detect components of interest.

The foregoing discussion discloses and describes many exemplary methods and embodiments of the present invention. As will be understood by those familiar with the art, the invention may be embodied in other specific forms without departing from the spirit or essential characteristics thereof. Accordingly, the disclosure of the present invention is intended to be illustrative, but not limiting, of the scope of the invention, which is set forth in the following claims.

1. A method for identifying protein-protein interactions, comprising:
crosslinking interacting proteins;
subjecting crosslinked proteins to a liquid chromatographic separation;
subjecting an effluent of the liquid chromatographic separation to mass spectrometry analysis for molecular weight determination of intact proteins and protein complexes in a first state and a second state, wherein in the first state, the effluent is analyzed under conditions that preserve crosslinks and, wherein in the second state, the effluent is analyzed under conditions that disrupt crosslinks; and
identifying components of a protein complex by plotting molecular weight data of the first state versus molecular weight data of the second state.

2. The method of claim 1, further comprising
collecting fractions from the liquid chromatographic separation;
selecting fractions of interest based on results obtained by plotting molecular weight data of the first state versus molecular weight data of the second state;
subjecting the fractions of interest to a peptide level mass spectrometry analysis; and,
identifying components of the protein complex.

3. The method of claim 2, wherein components of the protein complex are identified by integrating data from the mass spectrometry analysis for molecular weight determination of intact proteins and protein complexes, and data from the peptide level mass spectrometry analysis.

4. The method of claim 2, wherein the peptide level mass spectrometry analysis is a bottom-up LC-MS/MS analysis.

5. The method of claim 2, wherein the peptide level mass spectrometry analysis is an MALDI MS analysis.

6. The method of claim 1, further comprising isolating and concentrating a sub-proteomic fraction of crosslinked proteins prior to the liquid chromatographic separation.

7. The method of claim 1, wherein the liquid chromatographic separation is performed with a macroporous reverse phase material.

8. The method of claim 1, wherein the mass spectrometry analysis for molecular weight determination is performed with ESI-TOF MS or MALDI-TOF MS.

9. The method of claim 1, wherein the crosslinking is performed in vitro.

10. The method of claim 1, wherein the crosslinking is performed in vivo.

11. The method of claim 1, wherein crosslinks of the crosslinked protein are disrupted by a gas phase fragmentation method selected from the group consisting of collisionally induced dissociation (CID), IR Multiphoton Dissociation (IRMPD), Electron Transfer Dissociation (ETD), Electron Capture Dissociation (ECD), Metastable Ion Dissociation (MAID) and Surface Induced Dissociation (SID)

12. The method of claim 10, wherein the gas phase fragmentation is performed in an ionization chamber.

13. The method of claim 1, wherein the crosslinking is performed using a hetero-bifunctional crosslinking reagent.

14. The method of claim 13, wherein the hetero-bifunctional crosslinking reagent is sulfo-SFAD.

15. The method of claim 1, wherein the crosslinking is performed using a reversible home-bifunctional crosslinker.

16. The method of claim 15, wherein the reversible home-bifunctional crosslinker is selected from the group consisting of N-hydroxysuccinimide (NHS) esters and Bis[2-(Succinimidooxycarbonyloxy)ethyl]sulphone (BSOCOES).

17. The method of claim 16, wherein the reversible home-bifunctional crosslinker is BSOCOES.

18. A system for identifying protein-protein interactions, comprising:
a liquid chromatographic unit capable of high resolution separation of protein molecules;
a mass spectrometry (MS) unit coupled to the chromatographic unit wherein molecular weights are determined for intact proteins and protein complexes in an effluent of the liquid chromatographic unit under a first state and a second state, wherein in the first state, the effluent is analyzed under conditions that preserve crosslinks and, wherein in the second state, the effluent is analyzed under conditions that disrupt crosslinks; and
a data acquisition system capable of collecting a first state MS data and a second state MS data, plotting the first state MS data versus the second state MS data to detect components of a protein complex.

19. The system of claim 18, further comprising a second MS unit coupled to the chromatographic unit for peptide based-identification of proteins in chromatographic fractions.

20. The system of claim 19, wherein the data acquisition system is capable of collecting protein ID data from the second MS unit and integrating the first MS state data, the second MS state data, and the protein ID data to identify components of the protein complex.
